(19) United States
(12) Patent Application Publication
(10) Pub. No.: US 2012/0330098 A1
(43) Pub. Date: Dec. 27, 2012

(54) WORKING INSERT FOR AN ENDOSCOPIC HOLLOW SHANK INSTRUMENT

(75) Inventors: Frank KNODEL, Knittlingen (DE); Josef BARTOLIC, Karlsruhe (DE); Eberhard KÖRNER, Knittlingen (DE)

(73) Assignee: RICHARD WOLF GMBH, Knittlingen (DE)

(21) Appl. No.: 13/530,162

(22) Filed: Jun. 22, 2012

(30) Foreign Application Priority Data
Jun. 24, 2011 (DE) 10 2011 105 442.5

(51) Int. Cl. A61B 1/018

(52) U.S. Cl. 600/104

(57) ABSTRACT

A working insert for an endoscopic hollow shank instrument includes a shank, on whose outer side is arranged a monopolar working electrode axially displaceable relative to the shank. The working electrode is connected to the shank at a distal end section of the shank via a guide component which forms an electrical insulation between the working electrode and the shank.

### BACKGROUND OF THE INVENTION

The present invention relates generally to a working insert for an endoscopic hollow shank instrument.

Monopolar HF application technology is applied with a multitude of medical therapy procedures. Hereby, monopolar working electrodes are used for resection and vaporisation of body tissue as well as for coagulation. Such a working electrode is usually part of a working insert with viewing optics arranged in a metallic shank and is led to a treatment location via an endoscopic hollow shank instrument. The working electrode is surrounded by an electrically insulating encasing with the exception of its distal end serving for the application.

Apart from working inserts, with which the wire-like working electrode is led along the shank for the viewing optics, such working inserts are also known, with which the working electrode is arranged outside the shank for the viewing optics. It is particularly with the latter working inserts that there is the danger of a short circuit between the working electrode and the shank for the viewing optics or the metallic shank of the hollow shank instrument occurring on account of an incorrect operation or a damaged electrical insulation of the working electrode.

### BRIEF SUMMARY OF THE INVENTION

Against this background, it is an objective of a preferred embodiment of the present invention to provide a working insert of the type mentioned above, with a working electrode arranged on the outer side of a shank for observations optics, whereby this working insert is to offer an improved protection from an electric short circuit formation compared to working inserts which have been known until now.

This objective is achieved by a working insert for an endoscopic hollow shank instrument with a shank, on whose outer side a working electrode is axially displaceable relative to the shank is arranged. The working electrode is preferably connected to the shank at a distal end section of the shank via a guide component. The guide component preferably forms an electrical insulation between the working electrode and the shank. Advantageous further formations of the working insert of a preferred embodiment of the present invention are to be deduced from the subsequent description as well as the drawing. Hereby, according to a preferred embodiment of the present invention, the features specified in the dependent claims in each case per se, but also in a technically meaningful combination can further form the solution according to the independent claim(s) and according to a preferred embodiment of the present invention.

The operating insert according to a preferred embodiment of the present invention for an endoscopic hollow shank instrument includes a shank. An electronic or optical system and usefully suitable illumination means such as for example fibre optics or LEDs are arranged in this shank. The shank is formed by a straight metallic tube. An HF-electrode as a working electrode is arranged on the outer side of the shank in an axially displaceable manner relative to the shank. Hereby, it can, for example, be the case of an electrode used for resection, vaporisation or coagulation. The working electrode is designed in a wire-like manner and extends essentially over the whole length of the shank parallel to its longitudinal axis. The working electrode is connected at its proximal end to a HF-electricity connection. With the exception of the distal end region, the working electrode is encased by an electrical insulation. This is connected to the shank at the distal end section of the shank via a guide component, for the stabilisation of the working electrode. The guide component is usefully designed in a manner such that it permits a movement of the working electrode relative to the shank, for example, the working electrode can axially displace along the shank.

According to a preferred embodiment of the present invention, the guide component forms an electrical insulation between the working electrode and the shank for the optical system. Apart from the electrically insulating encasing of the working electrode, accordingly by way of the guide component, a further electric insulator is provided between the working electrode and the shank, by which means an undesired flow of current from the live working electrode to the electrically conductive shank of the optical system in the working insert is additionally effectively prevented. Even if the insulating encasing of the working electrode should be damaged in the region of the guide component, an undesired flow of current or a short circuit between the working electrode and the shank is prevented.

For forming an electrical insulator, at least one section of the guide component is formed of an electrical insulating material, for example, of an electrically non-conductive material. This section is typically arranged in a manner such that it prevents an electrically conductive connection from the working electrode to the shank. Preferably, however, one envisages the guide component being designed completely of an electrically non-conductive material. Hereby, the guide component can basically consist of all electrical insulating materials. Preferably, the guide component is however designed of plastic. The use of plastic as a material for the guide component is particularly advantageous if the guide component is fastened on the shank in a movable manner. Here, the plastic permits a comparatively low-friction movement of the guide component on the shank. The guide component can be manufactured in a particularly economical manner by way of the injection moulding method. The guide component is thus preferably an injection moulded part.

With a further preferred design of the working insert according to a preferred embodiment of the present invention, the guide component is fixedly connected to the working electrode. Accordingly, the guide component and the working electrode form a rigid unit. With this design, the guide component is movably connected to the shank of the optical system and to the working insert, for example, the guide component is displaceable on the shank in the longitudinal direction of the shank.

Advantageously, one envisages the guide component at least partly engaging around the shank. Hereby, for fastening the guide component on the shank, it is necessary for the guide component to engage around the shank by more than half the shank periphery. A section of the guide component which serves for fastening the guide component on the shank can preferably be designed in a fork-like manner and comprise two limbs forming a fork and extending around the shank in an angular region of more than 180°.

Apart from this, the guide component can also be designed in a manner such that it completely encompasses or engages around the shank. In this context, one design of the guide component is advantageously envisaged, with which a tubular guide channel for receiving the shank is formed on the guide component. Accordingly, the guide component has an open or closed guide, whose cross section corresponds to the outer cross section of the shank of the optical system. The optical system is guided on the shank with a low radial play by way of this guide.

Usefully, the working insert according to a preferred embodiment of the present invention comprises a handle arranged on the proximal side. The working electrode can be displaced relative to the shank with this handle. For this, the working electrode is releasably coupled to the handle and can be moved on the shank of the optical system by way of displacement in the distal and in the proximal direction. The handle at its distal end comprises a preferably metallic tubular guide for the working electrode, the guide running parallel to the shaft, in order to also proximally ensure a stable guiding of the working electrode on the outer side of the shank. This guide for the working electrode is arranged radially on the outer side of the shank and connected to the shank.

Advantageously, the working electrode is surrounded directly on the proximal side of the guide component by a reinforcement tube. The reinforcement tube accordingly extends directly in the direction of the guide for the working electrode, the guide being formed on the handle. The reinforcement tube which is preferably designed in a metallic manner also serves for stabilizing the working electrode and is displaceable within or along the shank of the working insert in the distal and proximal direction with the working electrode.

Usefully, the length of the reinforcement tube is dimensioned in a manner that with the displacement of the working electrode, it cannot contact the guide for the working electrode, the guide being formed on the handle side. Accordingly, a distance exists between the reinforcement tube and the guide which is formed on the handle and is for the working electrode, even if the working electrode with the reinforcement tube is proximally displaced to the greatest possible extent. This always existing distance between the reinforcement tube and the handle-side guide has the advantage that with a damage to the insulating encasing of the working electrode in the region of the guide component or of the reinforcement tube, current is not able to pass from the metallic reinforcement tube onto the likewise metallically designed handle-side guide and from there onto the handle. Additionally, by way of the selected short length of the reinforcement tube on the one hand and of the electrically insulating guide component on the other hand, there is the advantage that the complete capacitance of the resectoscope and of the working current resulting therefrom is reduced.

Advantageously, with the working insert according to a preferred embodiment of the present invention, one strives to guide the shank and the working electrode connected thereto, in a stable manner in the hollow shank instrument, via which instrument the working insert is led to the treatment region lying in the inside of the body. For this purpose, a radially projecting projection is preferably formed on the guide component. This projection advantageously forms a bearing-contact surface onto the inner wall of the hollow shank of the endoscopic hollow shank instrument. The dimension of the projection transversely to the longitudinal axis of the shank of the working instrument is usefully such that the shank of the working insert with the guide component which is fastened on the shank and is for the working electrode, is guided with little radial play in the hollow shank, in which the working insert is inserted. Foe example, the working insert although being able to be moved in an uninhibited manner in the hollow shank in the longitudinal direction, a movement of the working insert transversely to the longitudinal extension of the hollow shank is however only possible to a negligible extent.

Particularly advantageously, several components can be provided, whose projection formed thereon differs with regard to the dimensions, so that the working insert with a selection of the suitable projection is introduced in hollow shanks with different inner diameters in the manner described above. Apart from this, advantageously one can provide such guide components whose bearing-contact surfaces are adapted to the inner wall of these hollow shanks by way of differently designed projections, in dependence on the respective hollow shank.

One further advantageously envisages the working insert comprising an optical marking. This optical marking can represent a characterisation, which, for example, renders recognisable the electrode design, the suitability of the guide component for fastening on a shank with a certain shank diameter or the suitability of a working insert for application in a hollow shank of a hollow shank instrument with a certain inner cross section. It is very simply possible via different optical markings, to select a necessary working electrode or a necessary working insert. With regard to the optical marking, it is preferably the case of a colour marking Guide components which are formed from plastic can be particularly simply be manufactured from differently coloured plastics.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawing are shown:

FIG. 1 is a schematically simplified top perspective view of a working insert for an endoscopic hollow shank instrument in accordance with the present invention;

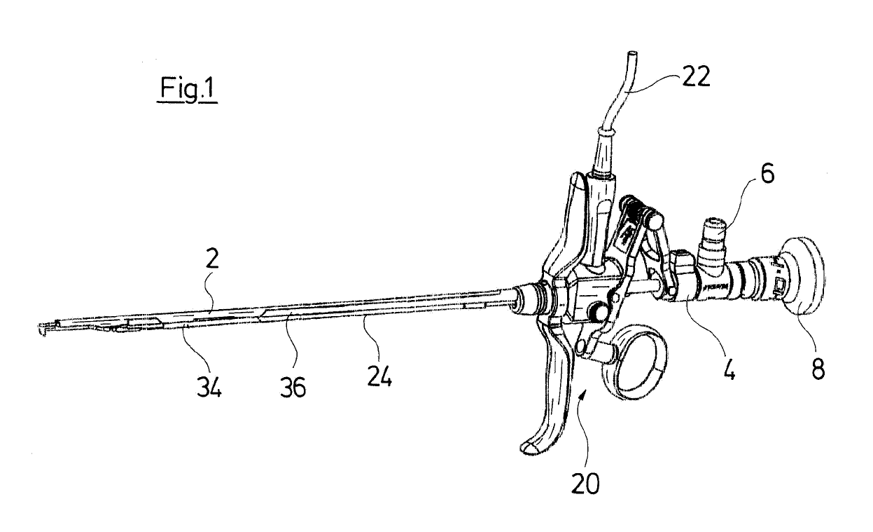

FIG. 2 is a schematically simplified perspective view of a first preferred embodiment of a distal end section of the working insert according to FIG. 1;

FIG. 3 is a schematically simplified perspective view of a second preferred embodiment of the distal end section of the working insert according to FIG. 1; and

FIG. 4 is in a schematically simplified sectioned view of a distal end section of an endoscopic hollow shank instrument with a working insert according to FIG. 1, which is arranged therein.

### DETAILED DESCRIPTION OF THE INVENTION

Certain terminology is used in the following description for convenience only and is not limiting. The word “outwardly” refers to a directions away from the geometric center of the device, and designated parts thereof, in accordance with the present invention. Unless specifically set forth herein, the terms “a,” “an” and “the” are not limited to one element, but instead should be read as meaning “at least one.” The terminology includes the words noted above, derivatives thereof and words of similar import.

Referring to the drawings in detail, wherein like numerals indicate like elements throughout the several views, the represented working insert preferably includes optics with an outer shank 2 which is preferably formed by a straight, rigid, metallic tube. An electronic or optical system for viewing a treatment region and illumination means for illuminating the treatment region are arranged in the shank 2. A coupling part 4 for receiving optics is preferably arranged on the proximal end of the working insert.

A monopolar working electrode 10 is preferably arranged on the outer side of the shank 2. The working electrode 10 is preferably directed parallel to the shank 2. With the represented working electrode 10, it is the case of a cutting electrode. The working electrode 10 preferably includes an electrode stem 12 which forks distally into two electrode limbs 14, 16, which are distanced to one another. An electrode wire 18 led through the electrode stem 12 and the electrode limbs 14, 16 leads from the distal end of the electrode limb 14 to the distal end of the electrode limb 16, wherein it is exposed between the distal ends of the electrode limbs 14, 16 and, thus, forms a cutter loop. The working electrode 10 on the outer side has an electrical insulation casing in the region of the electrode stem 12 as well as in the region of the electrode limbs 14 and 16.

On the proximal side, the working electrode 10 is preferably coupled to a handle 20 and is connected to a high frequency electricity connection 22 arranged on the handle 20. Moreover, the optical system with the shank 2 of the handle is guided through the handle 20. The working electrode 10 is displaceable along the shank 2 in the distal and proximal direction by way of the handle 20.

A tubular metallic guide 24 for the working electrode 10 is preferably formed at the distal end of the handle 20. This guide 24 forms a guide channel, through which the working electrode 10 is led. Moreover, the guide 24 is connected to the shank 2.

A guide component 26a, 26b is preferably rigidly fastened on the electrode stem 12, in the region of the working electrode 10, in which the electrode stem 12 merges into the two electrode limbs 14, 16, respectively. Hereby, the working electrode 10 or its electrode stem 12 are led through a channel which is formed on the component 26a, 26b and which peripherally surrounds the electrode stem 12. For guiding the working electrode 10 on the shank 2, the guide component 26a, 26b is connected to the shank 2 of the optical system, wherein the connection of the guide component 26a, 26b and the shank 2 is such that the guide component 26a, 26b although being fixed with a positive fit in the direction transversely to the shank 2, is however movable in the longitudinal direction of the shank 2.

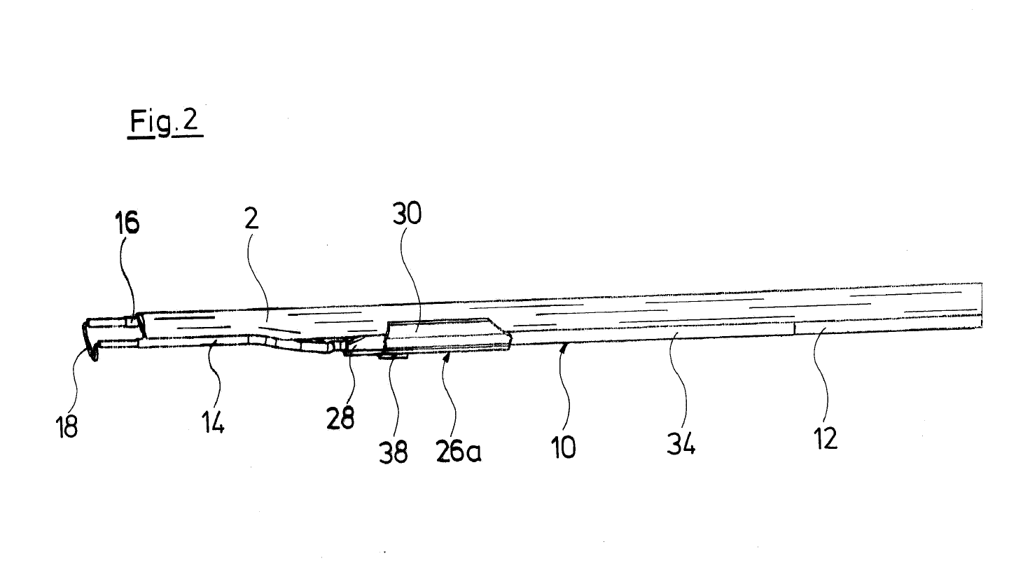

With the preferred embodiment example represented in FIG. 2, the guide component 26a outside a region 28, through which the working electrode 10 is led, preferably includes two laterally projecting limbs 30 which partly encompass or engage around the shank 2 at two opposite sides of the shank 2.

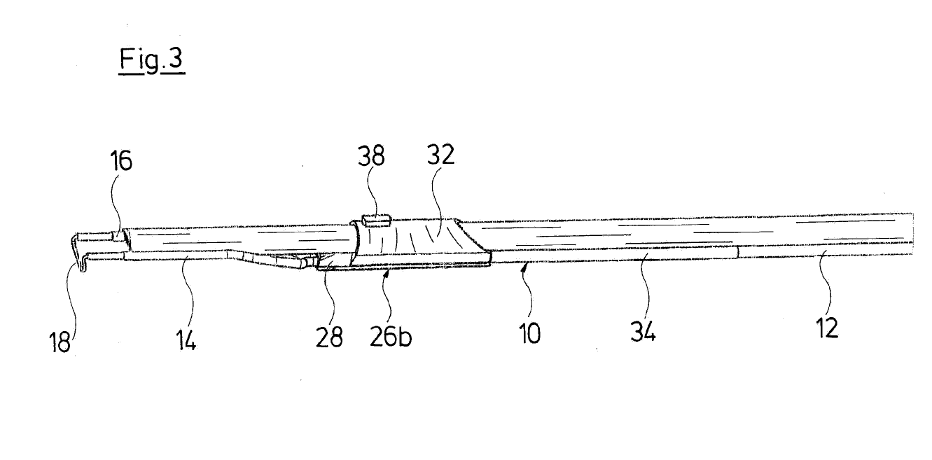

The guide component 26b, which is represented in FIG. 3, preferably engages around the shank 2 in a complete manner. Here, outside the region 28 of the guide component 26b, a region 32 is provided, which forms a closed guide channel for the shank 2, so that the shank 2 is peripherally completely surrounded by the guide component 26b.

The guide component 26a as well as the guide component 26b are preferably designed as a plastic injection moulded part, thus are to be seen as electrically non-conductive. Accordingly, the guide components 26a, 26b form electrical insulators which protect the working insert given a damage of the insulating casing of the working electrode 10 in the region of the guide component 26a, 26b, from an undesired flow of current from the working electrode 10 to the shank 2 forming an electrical conductor 2.

On the proximal side of the guide components 26a, 26b, the electrode stem 12 is surrounded in each case by a reinforcement tube 34. The reinforcement tube 34 which is fixedly connected to the working electrode 10 and is accordingly displaceable with the working electrode 10 in the distal and proximal direction, serves for stabilising the working electrode 10 and is designed in a metallic, this electrically conductive manner. The length of the reinforcement tube 34 is preferably dimensioned such that with its displacement with the working electrode 10 in the direction of the handle 20, it cannot contact the guide 24 of the handle 20. For example, a region 36, in which the electrode stem 12 surrounded by an electric insulation is exposed, always exists between the reinforcement tube 34 and the guide 24. Accordingly, a flow of current from the reinforcement tube 34 to the guide 24 and the handle 20 connecting proximally thereon cannot occur if there is damage to the electrical insulation of the electrode stem 12 within the reinforcement tube 34.

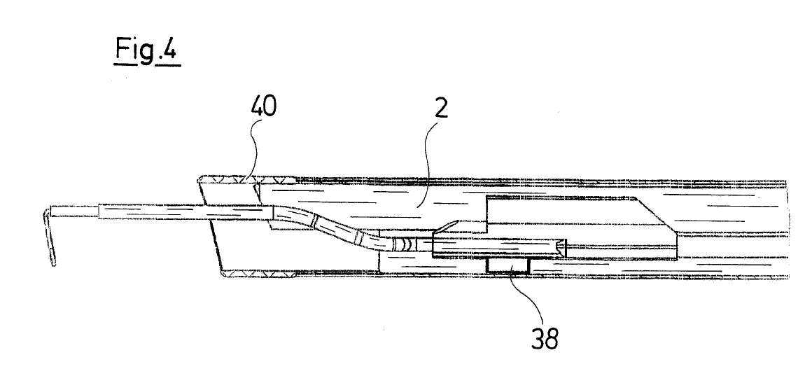

The guide component 26a as well as the guide component 26b each has a radially outwardly projecting projection 38. With the guide component 26a represented in FIG. 2, this projection 38 is arranged on the outer side of the region 28, while the projection 38 with the embodiment example represented FIG. 3 is arranged on the outer side of the region 32. As is to be deduced from FIG. 4, the outer side of the projection 38 forms a bearing-contact surface onto the inner wall of a hollow shank 40 of an endoscopic hollow shank instrument, via which the working insert is led to the region of treatment. The height of the projection 38 is selected in each case corresponding to the inner diameter of the hollow shank 40, into which the working insert is to be inserted, in a manner such that the shank 2 and the guide components 26a, 26b connected thereto on the outer side, in the hollow shank 40 only have a very low radial play to the inner wall of the hollow shank 40.

The guide components 26a, 26b each have a colour marking, with which, for example, a special electrode design or the suitability for a hollow shank of an endoscopic hollow shank instrument with a certain inner diameter is rendered directly recognisable. This, however, is not evident from the drawing.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A working insert for an endoscopic hollow shank instrument comprising a shank (2), a working electrode (10) being arranged on an outer side of the shank (2) and being axially displaceable relative to the shank (2), a guide component (26a, 26b) connecting the working electrode (10) to the shank (2) at a distal end section of the shank (2), the guide component (26a, 26b) forming an electrical insulation between the working electrode (10) and the shank (2).

2. A working insert according to claim 1, wherein the guide component (26a, 26b) is formed of an electrically non-conductive material.

3. A working insert according to claim 1, wherein the guide component (26a, 26b) consists of plastic.

4. A working insert according to claim 1, wherein the guide component (26a, 26b) is fixedly connected to the working electrode (10).

5. A working insert according to claim 1, wherein the guide component (26a, 26b) at least partly peripherally encompasses the shank (2).

6. A working insert according to claim 1, wherein a tubular guide channel for receiving the shank (2) of the optical system is formed on the guide component (26b).

7. A working insert according to claim 1, further comprising a handle (20) arranged on a proximal side of the working electrode (10), the working electrode (10) being displaceable with respect to the handle (20), the handle (20) at a distal end thereof comprising a tubular guide (24) for the working electrode (10).

8. A working insert according to claim 7, wherein the working electrode (10) is surrounded directly on the proximal side by a reinforcement tube (34), a length of the reinforcement tube (34) being dimensioned such that the reinforcement tube (34) does not contact the guide (24) formed on the handle side.

9. A working insert according to claim 1, wherein the guide component (26a, 26b) includes a radially projecting projection (38) which forms a bearing-contact surface onto an inner wall of a hollow shank (40) of the endoscopic hollow shank instrument.

10. A working insert according to claim 9, wherein the working insert has an optical marking.
